(19) United States
(12) Patent Application Publication
(10) Pub. No.: US 2004/0127871 A1
(43) Pub. Date: Jul. 1, 2004

(54) Secondary absorbent article

(76) Inventors: Thomas W. Odorzynski, Green Bay, WI (US); Connie J. Warshall, Neenah, WI (US)

Correspondence Address: KIMBERLY-CLARK WORLDWIDE, INC., 401 NORTH LAKE STREET, NEENAH, WI, 54956

(21) Appl. No.: 10/335,504

(22) Filed: Dec. 31, 2002

(51) Int. Cl. A61F 13/15; A61F 13/20

(52) U.S. Cl. 604/378

(57) ABSTRACT

A secondary absorbent article includes a liquid-permeable liner attached to an absorbent core that includes an absorbent layer. The absorbent layer includes a plurality of channels that extend through the absorbent layer. The secondary absorbent article optionally includes an attachment means for attaching the secondary absorbent article to a primary absorbent article. The geometry of the channels is designed to allow a portion of the insulting body fluids to flow through the absorbent core of the secondary absorbent article, while also retaining some body fluids in the absorbent core of the secondary absorbent article. The secondary absorbent article augments the absorbent capacity of the primary absorbent article while presenting a drier body-facing surface to the skin of the wearer.

### BACKGROUND OF THE INVENTION

The present invention generally relates to disposable secondary absorbent articles useful for bolstering the effectiveness of primary absorbent articles. More particularly, the present invention relates to disposable secondary absorbent articles useful when there is a need to increase the absorbent capacity of disposable primary absorbent articles such as disposable diapers.

Disposable diapers and incontinence products are designed to provide absorbent capacity that is sufficient for typical daytime use, for example two to three hours, by typical users for which the diaper is sized. Cost and size constraints prevent disposable diaper manufacturers from designing diapers with absorbent capacities large enough to handle insults that far exceed the average. However, there are times when extra absorbency is required, such as for nighttime use or other times when the product will be worn for periods of time longer than usual.

Absorbent inserts are known in the art. However, many of these inserts are designed to be utilized to full capacity before allowing body fluids to begin to fill the primary absorbent product. This allows the secondary product to be disposed of prior to any soiling of the primary product, thereby preserving the primary product for continued use and resulting in cost savings for the consumer. However, because the secondary product is in close proximity to the skin of the wearer, excessive fluid in this area can cause deterioration of skin health. Therefore, there is a need for a secondary absorbent article that works in conjunction with the primary absorbent article to provide extra absorbent capacity during those times when extra absorbent capacity is needed, and to do so while preserving skin health by minimizing wetness against the skin of the wearer.

### SUMMARY OF THE INVENTION

The aforementioned needs are addressed by the present invention which provides a disposable secondary absorbent article. In one aspect of the present invention, the secondary absorbent article includes a liquid-permeable liner attached to an absorbent core that includes an absorbent layer that includes a plurality of channels that extend through the absorbent layer. In one embodiment, the absorbent layer has a channel density between about 0.25 and about 20 channels per square centimeter. In another embodiment, the absorbent layer has a channel density between about 1 channel per square centimeter and about 10 channels per square centimeter. In even another embodiment, the absorbent layer has a channel density between about 2 channels per square centimeter and about 5 channels per square centimeter. Further, the channels may be substantially parallel, and/or may be substantially cylindrical in shape, having diameter between about 1 millimeter and about 10 millimeters, or between 4 millimeters and about 6 millimeters.

In another embodiment, the channels have a cross-sectional area between about 1 and about 100 square millimeters. In a further embodiment, the total cross-sectional area of the channels at the external surface of the absorbent layer is between about 5% and about 30% of the external surface area of the absorbent layer. In an even further embodiment, the absorbent layer includes hollow projections that form an extension of the channels.

In another embodiment, the liner may comprise a spunbond material having a basis weight of between 10 grams per square meter and 34 grams per square meter. The liner may fully enclose the absorbent core. In a further embodiment, the absorbent core includes a tissue material wrapped about the absorbent layer. In an even further embodiment, the absorbent core includes more than one absorbent layer.

In another aspect, the present invention provides an absorbent article that includes a first liquid-permeable liner, a second liquid-permeable liner, and, an absorbent core. The second liquid-permeable liner has an exterior surface with an attachment means secured thereto. The attachment means is adapted to be adhered to a primary absorbent article. The absorbent core includes an absorbent layer that includes a plurality of channels. The channels extend through the absorbent layer, having a channel density between about 1 and about 20 channels per square centimeter. The channels have a cross-sectional area between about 1 and about 25 square millimeters, and the total area of the channels does not exceed about 30% of the external surface area of the absorbent layer. The absorbent core is enclosed by the first and second liners.

In another aspect, the present invention provides an absorbent article that includes a liquid-permeable body-side liner, a liquid-permeable garment-side liner, and an absorbent core including an absorbent layer. The absorbent layer includes a plurality of channels wherein the channels extend through the absorbent layer. The absorbent core is enclosed by the body-side liner and the garment-side liner. The garment-side liner includes a roughened surface.

In another aspect, the present invention includes an absorbent system that includes a secondary absorbent article that is attached to a primary absorbent article. The secondary absorbent article includes a liquid-permeable liner attached to an absorbent core that includes an absorbent layer that includes a plurality of channels that extend through the absorbent layer. Desirably, the shape of the primary absorbent article is substantially the same as the shape of the secondary absorbent article. In one embodiment, the absorbent capacity of the primary absorbent article substantially matches the absorbent capacity of the secondary absorbent article.

Other features and aspects of the present invention are discussed in greater detail below.

### BRIEF DESCRIPTIONS OF THE DRAWINGS

FIG. 1 is a top view of a secondary absorbent article.

FIG. 2 is a cross-sectional view of the secondary absorbent article shown in FIG. 1 taken along line 2-2.

FIG. 3 is a bottom view of the secondary absorbent article shown in FIG. 1.

FIG. 4 is a perspective view of the secondary absorbent article positioned in the crotch portion of a primary absorbent article.

FIG. 5 is a cross-sectional view of the combination secondary absorbent article and primary absorbent article shown in FIG. 4 taken along line 5-5.

FIG. 6 is a perspective view of an absorbent layer material.

### DETAILED DESCRIPTION

The invention will now be described in detail with reference to particular embodiments thereof. The embodiments are provided by way of explanation of the invention, and not meant as a limitation of the invention. For example, features described or illustrated as part of one embodiment may be used with another embodiment to yield still a further embodiment. It is intended that the present invention include these and other modifications and variations as come within the scope and spirit of the invention.

As used herein and in the claims, the term “comprising” is inclusive or open-ended and does not exclude additional unrecited elements, compositional components, or method steps. Accordingly, the term “comprising” encompasses the more restrictive terms “consisting essentially of” and “consisting of.”

The present invention provides a disposable secondary absorbent article that includes a body-side liner, an absorbent core, and, optionally, a garment-side liner. The absorbent core comprises an absorbent layer that has a plurality of open channels that extend from one exterior surface of the absorbent layer to the opposite exterior surface of the absorbent layer. The channels facilitate the passage of fluid from the body-side liner to and through the optional garment-side liner and into the target zone of a primary absorbent article. The disposable secondary absorbent article is sized to fit within the target zone of a primary absorbent article. As used herein, the term “disposable” refers to articles which are intended to be discarded after a limited use and that are not intended to be laundered or otherwise restored for reuse.

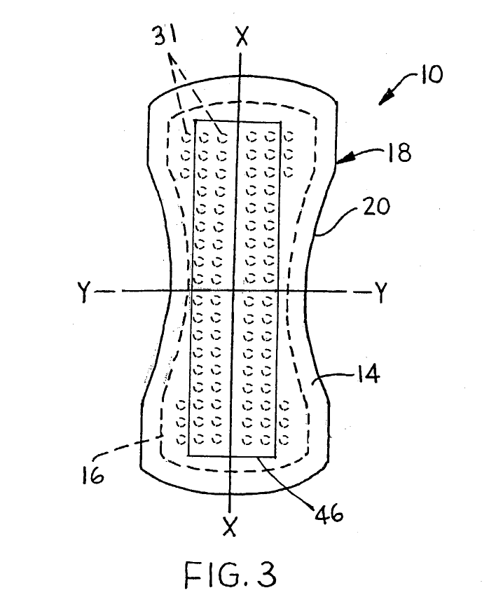

Referring to FIGS. 1-3, a disposable secondary absorbent article 10 is shown constructed of a body-side liner 12, an optional garment-side liner 14 and an absorbent core 16. The absorbent core 16, the body-side liner 12, and the garment-side liner 14 form an absorbent pad 18 having a crotch portion 20. The secondary absorbent article 10 is an elongated member having a longitudinal axis x-x, a transverse axis y-y and a vertical axis z-z. The secondary absorbent article 10 can have a rectangular, hourglass, race track, oval, elliptical or other geometrical configuration when viewed from the top. The secondary absorbent article 10 has an overall length measured parallel to the longitudinal axis x-x. In some embodiments, the secondary absorbent article 10 has an overall length of less than about 600 millimeters (mm). In other embodiments, the secondary article 10 may have an overall length of less than about 500 mm, less than about 400 mm, less than about 300 mm, or less than about 200 mm. The secondary absorbent article 10 also has a crotch width measured parallel to the transverse axis y-y. In some embodiments, the secondary article 10 has a crotch width of less than about 200 mm. In other embodiments, the crotch width of the secondary article 10 may be less than about 175 mm, less than about 150 mm, less than about 125 mm, less than about 100 mm, or less than about 75 mm. It should be noted that the secondary absorbent article 10 could have a wider width when measured away from the crotch portion. The secondary absorbent article 10 also has an overall height measured parallel to the vertical axis z-z. In some embodiments, the secondary absorbent article 10 has an overall height of from about 2 mm to about 25 mm. In other embodiments, the height of the secondary absorbent article 10 may be less than about 18 mm. Desirably, the secondary absorbent article 10 is sized to fit within a primary absorbent article. All of the above lengths, widths, and height dimensions are measured with respect to the outer edges of the liners 12, 14.

The body-side liner 12 suitably presents a bodyfacing surface that is compliant, soft feeling, and nonirritating to the skin of the wearer. The body-side liner 12 can be constructed from natural or synthetic material. The body-side liner 12 can be formed from a woven material, nonwoven material, finely perforated film, net material, porous foams, reticulated foams, and so forth. Suitable materials include, but are not limited to, fibrous materials such as bonded carded webs, meltblown fabrics, and spunbond fabrics. Suitable fibers include, but are not limited to, polyester, polypropylene, polyethylene, nylon or other heat-bondable fibers. Other polyolefins, such as copolymers of polypropylene and polyethylene, and linear low-density polyethylene also work well. A desirable material is a spunbond fabric. Spunbond material is a nonwoven material formed, for example, from polypropylene fibers. Spunbond is sold commercially by Kimberly-Clark Corporation having an office at 401 North Lake Street, Neenah, Wis. 54956. The spunbond material can contain from about 1% to about 2% titanium dioxide pigment to give it a clean, white appearance. The production of spunbonded nonwoven webs is illustrated in patents such as Appel, et al., U.S. Pat. No. 4,340,563, Dorschner et al., U.S. Pat. No. 3,692,618; Kinney, U.S. Pat. Nos. 3,338,992 and 3,341,394; Levy, U.S. Pat. No. 3,276,944; Peterson, U.S. Pat. No. 3,502,538; Hartman, U.S. Pat. No. 3,502,763; Dobo et al., U.S. Pat. No. 3,542,615; Matsuki et al., U.S. Pat. No. 3,802,817; and Harmon, Canadian Patent Number 803,714, the entire contents of these patents being incorporated herein by reference.

The body-side liner 12 can be formed from materials having a range of basis weights. When the body-side liner 12 is formed from a spunbond material, the material may have a basis weight of from about 10 grams per square meter (gsm) to about 34 gsm. Desirably, the basis weight for the spunbond material is from about 12 gsm to about 17 gsm.

The body-side liner 12 is liquid and vapor permeable. By “liquid and vapor permeable” it is meant that body fluids, especially urine, and vapors can pass therethrough. The body-side liner 12 is designed to allow body fluid, particularly urine, to quickly pass therethrough and be received by the absorbent core 16. The body-side liner 12 is suitably employed to help isolate the skin of the wearer from liquids held in the absorbent core 16. Because the body-side liner 12 is to be placed in contact with the genital area of a human body, the body-side liner 12 is capable of passing body fluid, voluntarily or involuntarily expelled from the urethra, downwardly into the absorbent core 16.

The body-side liner 12 can be constructed with pores or openings that permit liquids and vapors to pass therethrough. Additionally and/or alternatively, the material from which the body-side liner 12 is constructed can be treated to be hydrophilic. The body-side liner 12 is desirably less hydrophilic than the absorbent core 16 to present a relatively dry surface against the skin of the wearer, and is desirably sufficiently porous to be liquid and vapor permeable, permitting bodily fluids to readily penetrate through its thickness. A suitable treatment includes, but is not limited to, treatment with a surfactant commercially available from Uniqema Inc., a division of ICI of New Castle, Del., U.S.A., under the trade designation AHCOVEL Base N-62. The surfactant may be applied by any conventional means, such as spraying, printing, brush coating or the like. The surfactant may be applied to the entire body-side liner 12 or may be selectively applied to particular sections of the body-side liner 12, such as along the longitudinal centerline of the secondary absorbent article 10, to provide greater wettability of such sections.

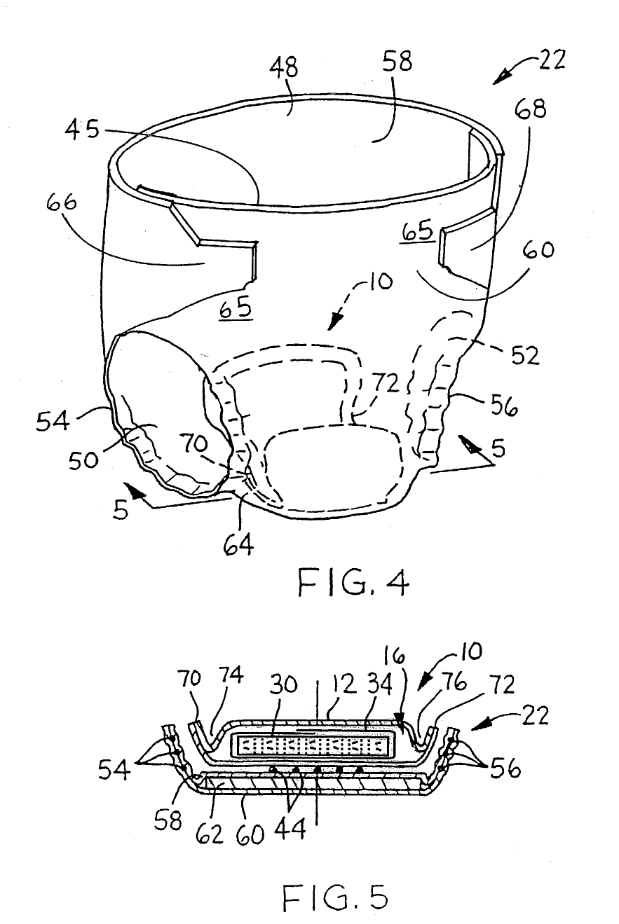

The optional garment-side liner 14, like the body-side liner 12, is designed to allow body fluid, particularly urine, and vapor to pass quickly therethrough. The garment-side liner 14 is designed to be positioned away from the human body. The garment-side liner 14 is capable of allowing body fluid exiting from the absorbent core 16 to pass downwardly into a primary absorbent article 22, such as that depicted in FIG. 4. The garment-side liner 14 can be constructed from natural or synthetic material. The garment-side liner 14 can be formed from a nonwoven material such as a spunbond material. The garment-side liner 14 can be formed from materials having a range of basis weights. When the garment-side liner 14 is formed from a spunbond material, the material may have a basis weight of from about 10 gsm to about 34 gsm. Desirably, the basis weight of the spunbond material is from about 12 gsm to about 17 gsm.

The material from which the optional garment-side liner 14 is constructed may be treated to be hydrophilic. Additionally and/or alternatively, the garment-side liner 14 can be constructed to have pores or openings that permit liquids and vapors to pass through. It should be noted that the body-side liner 12 and the garment-side liner 14 can be formed from the same material. When the body-side liner 12 and the garment-side liner 14 are the same material, they can optionally be a single piece of material wrapped around the absorbent core 16. Desirably, both the body-side liner 12 and the garment-side liner 14 are formed from a spunbond material and have a basis weight of about 12 gsm to about 17 gsm. The liquid permeability of the body-side liner 12 and the garment-side liner 14 can be equal to one another or be different from each other. Desirably, the liquid permeability of the garment-side liner 14 is at least equal to the liquid permeability of the body-side liner 12 to avoid pooling of the liquid within the absorbent core 16.

The body-side liner 12 and the optional garment-side liner 14 can be secured at their edges around the absorbent core 16 by an adhesive 28, which is desirably a construction adhesive. The construction adhesive 28 can be either a hot melt adhesive or a cold melt adhesive. A hot melt adhesive that works well is REXTAC® RT 2730. This construction adhesive is commercially available from Huntsman Polymers Corporation having a mailing address of 3040 Post Oak Blvd., Houston, Tex., 77056. It should be noted that the construction adhesive 28 could also be present at other locations within the secondary absorbent article 10. For example, the construction adhesive 28 can be present between the body-side liner 12 and the absorbent core 16. The construction adhesive 28 can also be present between the garment-side liner 14 and the absorbent core 16. If the body-side liner 12 and the garment-side liner 14 are a single piece of material wrapped about the absorbent core 16, the adhesive 28 can be present between layers of the material to prevent the material from unwrapping.

Other methods of securing the body-side liner 12 to the optional garment-side liner 14 are known to those skilled in the art. Non-limiting examples of other attachment methods include ultrasonic bonding, pressure bonds, heat bonds, heat and pressure bonds, thermal bonds wherein one material is heated above its melting temperature and is bonded to a second material, and so forth. The body-side liner 12 and the garment-side liner 14 can also be secured together by using thread if desired.

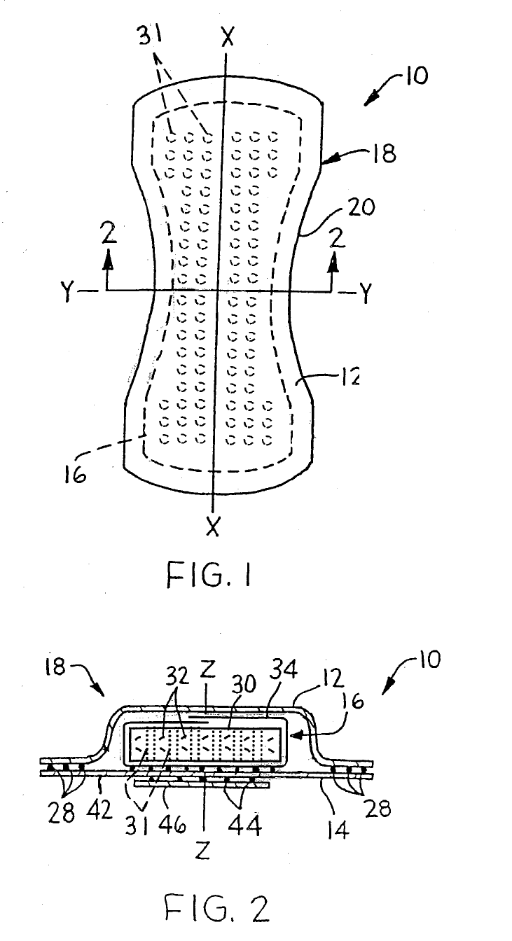

Referring now to FIG. 2, the absorbent core 16 can consist of one or more absorbent layers. The absorbent core 16 may be any absorbent material known to one skilled in the art that is suitable for absorption of bodily fluids such as urine and watery feces. The absorbent core 16 can be made from natural or synthetic fibers, including cellulose fibers, surfactant-treated meltblown fibers, wood pulp fibers, regenerated cellulose or cotton fibers, coform materials, combinations thereof, and so forth. Two desired absorbent materials are wood pulp fluff and coform material. Coform material is a blend of pulp and synthetic meltblown fibers. Lau, U.S. Pat. No. 4,818,464 and Anderson et al., U.S. Pat. No. 4,100,324, the entire contents of these patents being incorporated herein by reference, describe coform air laying processes that incorporate pulp fibers into meltblown fibers.

In FIG. 2, the absorbent core 16 is shown having a single absorbent layer 30. The absorbent layer 30 is positioned below the body-side liner 12. The absorbent layer 30 has a plurality of channels 31 that extend from the external surface of the absorbent layer 30 that is closest to the body-side liner 12 to the external surface of the absorbent layer 30 that is closest to the optional garment-side liner 14. The channels 31 are desirably substantially parallel and are desirably oriented perpendicular to the surfaces of the absorbent layer 30 to allow at least a substantial portion of the body fluids to flow unimpeded from the body-side liner 12 to the garment-side liner 14 and into the absorbent core of the primary absorbent article 22. The sides of the channels 31 may be angled to funnel bodily fluid from the external surface of the absorbent layer 30 that is closest to the body-side liner 12 to the external surface of the absorbent layer 30 that is closest to the garment-side liner 14.

The density of the channels 31 and cross-sectional area of the channels 31 are selected to control the quantity of bodily fluids that flow through the absorbent layer 30 relative to the quantity of bodily fluids that are absorbed by the absorbent layer 30. The channel density is defined as the number of channels per unit surface area of the absorbent layer 30. In some embodiments, the absorbent layer 30 has a channel density between about 0.25 channels per square centimeter and about 20 channels per square centimeter. In other embodiments, the absorbent layer 30 may have a channel density between about 1 channel per square centimeter and about 10 channels per square centimeter, or between about 2 channels per square centimeter and about 5 channels per square centimeter. In some embodiments, the channels 31 have a cross-sectional area between about 1 and about 100 square millimeters. In some embodiments, the total cross-sectional area of the channels 31 at the external surface of the absorbent layer 30 is between about 5% and about 30% of the external surface area of the absorbent layer 30. In other embodiments, the external surface of the absorbent layer 30 is between about 10% and about 30%, between about 15% and about 30%, between about 20% and about 30%, or between about 25% and about 30% of the external surface area of the absorbent layer 30. Greater channel density and larger channel cross-sectional area will increase the relative quantity of bodily fluids that will transfer through the absorbent layer 30 as opposed to being absorbed by the absorbent layer 30. Lower channel density and smaller channel cross-sectional area will decrease the relative quantity of bodily fluids that will transfer through the absorbent layer 30 as opposed to being absorbed by the absorbent layer 30.

The channels 31 may have any shape through which fluid will pass, for example, round, cylindrical, square, rectangular, triangular, pentagonal, hexagonal, and so forth. When the channels 31 have a round shape, the diameter of the round channels desirably ranges from about 1 millimeter to about 15 millimeters. In other embodiments, the diameter of the round channels may range from about 1 millimeter to about 10 millimeters, or from about 4 millimeters to about 6 millimeters.

The absorbent layer 30 may be divided into zones that may have different channel geometries to provide different fluid absorbency and “flow through” characteristics. In those areas of the secondary absorbent article 10 where it is desired that a greater portion of the bodily fluids be transferred through the secondary absorbent article 10 to the primary absorbent article 22, there may be a greater number or density of channels 31, the channels 31 may have a larger cross-sectional area, or both. Similarly, in those areas of the secondary absorbent article 10 where it is desired that a greater portion of the bodily fluids be retained within the secondary absorbent article 10, there may be fewer channels 31, the channels 31 may have a smaller cross-sectional area, or both. The channels 31 may have any combination of density and cross-sectional area that will not destroy the structural integrity of the absorbent layer 30.

The channels 31 may be formed in the absorbent layer 30 during initial formation of the material used for the absorbent layer 30, or may be created in the absorbent layer 30 in a processing step that occurs after formation of the material used for the absorbent layer 30. Materials used for the absorbent layer 30 may be formed on forming fabrics. The channels 31 may be created in the material by utilizing forming fabrics that have a plurality of projections having a cross-sectional shape, size, and density that matches the desired shape, size, and density of the channels in the absorbent layer 30. The absorbent material is formed around the projections such that when the absorbent material is removed from the forming fabric the resultant absorbent material has a plurality of channels 31. A similar forming process for making apertured nonwoven materials is disclosed in Griesbach III et al., U.S. Pat. No. 5,575,874, the entire contents of this patent being incorporated herein by reference.

Alternatively, there are a number of processes useful to create channels 31 in absorbent material that has already been formed. Such processes physically punch channels 31 into the absorbent layer 30. Examples include, but are not limited to, needle punching assemblies, embossing roll assemblies, intermeshing roll assemblies which have three-dimensional projections to displace fibers away from the projections, and so forth. One exemplary process for creating channels in a fibrous web using nipped anvil and pattern rolls rotating at different speeds is described in Majors et al., U.S. Pat. No. 5,704,101, the entire contents of this patent being incorporated herein by reference.

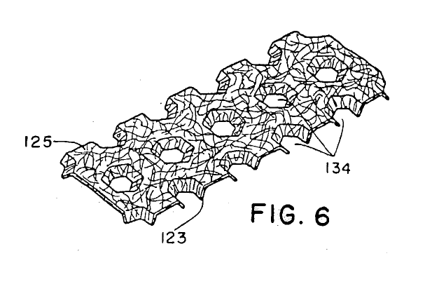

Some processes for creating channels 31 in an absorbent material may create three dimensional hollow projections that form an extension of the channels 31 on one side of the absorbent material. Referring to FIG. 6, a portion of an absorbent layer 125 is shown having hexagonally-shaped channels 134 that extend through projections 123 that protrude from one side of the absorbent layer 125. Desirably, in the secondary absorbent article 10 these projections 123 are oriented towards the garment-side liner 14. By orienting the projections 123 in this manner, bodily fluid at the body-side liner surface of the absorbent layer 125 is funneled into the channels 134 and is more readily transferred to the absorbent core of the primary absorbent article 22 (see FIG. 4). Engelbert et al., U.S. Pat. No. 4,741,941, the entire contents of this patent being incorporated herein by reference, describes a process for generating channels 134 with projections 123 in fibrous materials.

Referring again to FIG. 2, the absorbent layer 30 can optionally contain a high-absorbency superabsorbent material 32. Suitable high-absorbency materials for the absorbent layer 30 include, but are not limited to, natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces. Examples of suitable synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include, but are not limited to, natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. The superabsorbent material 32 that can be added to the absorbent core 16 can be produced to have almost any physical form. Commonly, the superabsorbent material 32 is in the shape of small particles having a major dimension of less than about 500 microns. The superabsorbent material 32 may also be, for example, in the form of fibers, flakes, rods, spheres, needles, or the like. Such high-absorbency materials are well known to those skilled in the art and are widely commercially available. Superabsorbent materials are commercially available from several different vendors including Dow Chemical Company and Stockhausen Inc. Two superabsorbents that work well for retaining urine are DRYTECH® 2035M and FAVOR® SXM 880. DRYTECH® 2035M is available from Dow Chemical Company having a mailing address of 2030 Dow Center, Midland, Mich., 48674. FAVOR® SXM 880 is available from Stockhausen Inc. having a mailing address of 2401 Doyle Street, Greensboro, N.C., 27406.

The superabsorbent material 32 is normally added to the absorbent layer 30 to increase the amount of fluid that the absorbent core 16 can absorb and retain. The fluid retention capacity, also referred to as fluid absorbent capacity, of the absorbent core 16, for urine, is desirably at least 50 grams (g). In some embodiments, the fluid absorbent capacity of the absorbent core 16 for urine may be at least 100 g, may be at least 150 g, or may be at least 200 g. In some embodiments, the capacity of the absorbent core 16 is selected to approximately match that of a particular or corresponding type or size of a primary absorbent article. In other embodiments, the capacity of the absorbent core 16 can be a percentage, for example, 20%, 40%, 60%, or 80%, of the capacity of a particular or corresponding type or size of a primary absorbent article. In even other embodiments, the absorbent capacity of the absorbent core 16 can be greater than that of a particular or corresponding type or size of a primary absorbent article. It should be noted that saline is normally used in place of human urine when measuring the fluid retention or absorbent capacity of the absorbent core 16. Saline is an aqueous solution of about 0.9% sodium chloride by weight. One brand of saline is S/P® Certified Blood Saline, which is commercially available from Baxter Diagnostics having an office in McGraw Park, Ill. The reason for using saline instead of actual human urine is that it is sanitary to handle in a laboratory. In addition, the absorbency results for an absorbent core tested using about 0.9% saline is very close to the absorbency results using human urine.

The superabsorbent material 32 may be substantially homogeneously mixed within the absorbent layer 30 or may be nonuniformly mixed. The fluff and superabsorbent material 32 may also be selectively placed into desired zones of the absorbent core to better contain and absorb body exudates. The concentration of the superabsorbent material 32 may also vary through the thickness of the absorbent layer 30. Alternatively, the absorbent core 16 may comprise a laminate of fibrous webs and superabsorbent material or other suitable means of maintaining a superabsorbent material in a localized area.

Returning to FIG. 2, the secondary absorbent article 10 is shown including an optional tissue 34 that is C-folded around the absorbent layer 30. The tissue 34 is typically placed about the absorbent core 16 over at least the two major facing surfaces thereof and may be composed of an absorbent cellulosic material, such as creped wadding or a high wet-strength tissue. In accordance with one embodiment of the invention, the tissue wrapsheet can be configured to provide a wicking layer that helps to rapidly distribute liquid over the mass of absorbent materials comprising the absorbent core 16. The edges of the tissue 34 on one side of the absorbent fibrous mass may be bonded to the edges of the tissue wrapsheet located on the opposite side of the absorbent fibrous mass to effectively entrap the absorbent core 16. The tissue 34 functions to retain the superabsorbent material 32 therein. It is advantageous to utilize at least about 15 percent by weight of superabsorbent material 32 in the absorbent layer 30. Desirably, at least about 20 percent by weight of the superabsorbent material 32 is present in the absorbent layer 30. More desirably, about 24 percent by weight of the superabsorbent material 32 is present in the absorbent layer 30. The use of this percentage by weight of the superabsorbent material 32 will significantly increase both the fluid absorbing and the fluid retention capacity of the absorbent core 16.

It should be noted the superabsorbent material 32 does not have to be present in the absorbent layer 30. However, one must recognize that the absorbent capacity of the absorbent layer 30 will be less if no superabsorbent material 32 is present. When one uses a concentration of superabsorbent material 32 in the absorbent layer 30 in excess of about 15 percent by weight, it is advantageous to wrap the absorbent layer 30 in the tissue 34 to prevent fine superabsorbent particles from falling out. If no superabsorbent material 32 is present, or if the superabsorbent material 32 represents less than about 15 percent by weight of the absorbent layer 30, then the tissue 34 could be eliminated.

It should be noted that even though the absorbent core 16 is described as including a single absorbent layer 30, it could be formed from two or more absorbent layers. Additional absorbent layers may be constructed of the same or different components as the absorbent layer 30. For example, one absorbent layer may be constructed of a coform material, while a second layer may be constructed of wood pulp only. As another example, one absorbent layer may contain a first type of superabsorbent particles while a second absorbent layer may contain a second type of superabsorbent particles or no superabsorbent particles at all. Many suitable combinations of components are possible.

The absorbent core 16 may have any suitable shape. Exemplary shapes include rectangular, I-shaped, T-shaped, and so forth. Optionally, the absorbent core 16 is narrow in the crotch portion 20 of the secondary absorbent article 10. In some embodiments, the absorbent core 16 has a crotch width of less than about 200 mm. In other embodiments, the crotch width of the absorbent core 16 may be less than about 175 mm, less than about 150 mm, less than about 125 mm, less than about 100 mm, or less than about 75 mm. Generally, the width of the absorbent core 16 is a measurement of the lateral distance between opposing side edges of the absorbent core 16 at the narrowest point of the absorbent core 16 along the longitudinal axis. The narrow width of the absorbent core 16 in the crotch portion 20 allows the absorbent core 16 to better fit into a primary absorbent article and to better fit between the legs of the wearer. It is apparent that the dimensions and the absorbent capacity of the absorbent core 16 desirably correspond to the size of the intended wearer and the liquid loading imparted by the intended use of the secondary absorbent article 10.

Still referring to FIG. 2, the absorbent core 16 has an overall height measured parallel to the vertical axis z-z. In some embodiments, the absorbent core 16 has an overall height of from about 2 mm to about 25 mm. In other embodiments, the height of the absorbent core 16 may be less than about 18 mm. As the height of the absorbent core 16 increases, the fluid capacity of the absorbent core 16 normally increases. However, one should understand that the materials from which the absorbent core 16 is constructed, the presence of the superabsorbent material 32, and the thickness of the absorbent core 16 will all determine the fluid capacity of the absorbent core 16.

Still referring to FIG. 2, the optional garment-side liner 14 of the secondary absorbent article 10 has an exterior surface 42. Secured to the garment-facing surface 42 is an optional attachment means 44, the purpose of which is to allow attachment of the secondary article 10 to the primary article 22. In other embodiments, there may be more than one attachment means 44 attached to the garment-facing surface 42. Those skilled in the art will appreciate that more than one attachment means 44 may be positioned in any number of configurations that will provide adequate attachment of the secondary article 10 to the primary absorbent article 22. For example, there may be one attachment means attached to the garment-facing surface 42 at one end of the secondary article 10 and a second attachment means attached to the garment-facing surface 42 at the opposite end of the secondary article 10. In yet another embodiment, if no garment-side liner 14 is used, the optional attachment means 44 may be attached to the absorbent core 16.

The optional attachment means 44 is desirably one or more strips of a garment adhesive. However, the attachment means 44 can include other forms of attachment mechanisms. Other forms of attachment mechanisms that can be utilized include hook and/or loop fasteners, tape, glue, etc. A VELCRO® fastener is one form of a hook fastener that engages a loop material. VELCRO® is a registered trademark of Velcro Industries having a mailing address of 406 Brown Avenue, Manchester, N.H. 03103. When the attachment means 44 is a garment adhesive, the adhesive can be either a hot or cold melt adhesive that is sprayed, brushed, slot coated or otherwise applied onto the exterior surface 42 of the garment-side liner 14. As one example, the garment adhesive can be applied as one or more beads, lines or strips of adhesive aligned approximately parallel to the longitudinal axis x-x. Desirably, the garment adhesive is a hot melt adhesive. Garment adhesive is commercially available from several vendors. One such vendor is National Starch and Chemical Company having an office at 10 Finderne Avenue, Bridgewater, N.J. 08807.

When a hook and loop fastener is used as the optional attachment means 44, the hook portion can be secured to a portion of the garment-side liner 14 and the loop portion can be secured to a portion of the primary absorbent article 22 or vice versa. It should also be noted that some materials, like spunbond materials, can serve the same function as a loop material and therefore a separate patch of loop material does not have to be secured opposite to the hook material. For example, if a patch of hook material is secured to the garment-side liner 14, the primary absorbent article 22 will not be required to have a patch of loop material secured to it.

As mentioned above, the optional garment-side liner 14 has an exterior surface 42 and a portion of this exterior surface 42 will contact the primary absorbent article 22. The portion of the exterior surface 42 that will contact the primary absorbent article 22 can be formed to have a high coefficient of friction making it a non-skid surface. For example, the exterior surface 42 can consist of a roughened surface, a treated surface or be made from a non-skid material. The roughened, treated or non-skid surface will provide a physical attachment to the primary absorbent article 22. Another way of stating this is to say that the non-skid portion of the exterior surface 42 serves a similar function as the attachment means 44.

The optional attachment means 44 can be protected from contamination by a releasable or removable peel strip 46. The peel strip 46 is designed to be removed by the consumer just prior to positioning and attaching the secondary absorbent article 10 to the crotch portion of the primary absorbent article 22. The releasable peel strip 46 is generally slightly larger in overall dimensions when compared to the attachment means 44 so as to enable the secondary absorbent article 10 to be manufactured at high speeds. By “high speeds” it is meant the ability to manufacture at a speed of more than 200 secondary absorbent articles per minute. The peel strip 46 can be a white Kraft paper, coated on one side so that it can be easily released from the attachment means 44.

In accordance with one embodiment of this invention, the secondary absorbent article 10 optionally may include a pair of containment flaps that are configured to provide a barrier to the lateral flow of body exudates. The containment flaps may be located along the laterally opposing side edges of the secondary absorbent article 10 adjacent the side edges of the absorbent core 16. Each containment flap typically defines an unattached edge that is configured to maintain an upright, perpendicular configuration in at least the crotch region of the secondary absorbent article 10 to form a seal against the body of the wearer. Each containment flap may extend longitudinally along at least a portion of a length of the absorbent core 16. Desirably, each containment flap extends along substantially the entire length of the absorbent core 16 to better contain the body exudates. Such containment flaps are generally well known to those skilled in the art. For example, suitable constructions and arrangements for containment flaps are described in Enloe, U.S. Pat. No. 4,704,116, the entire contents of this patent being incorporated herein by reference. In another embodiment, the edges of the body-side liner 12 and/or the garment-side liner 14 may be elasticized to form a containment flap as described above.

Referring now to FIGS. 4 and 5, a primary absorbent article 22 is shown having a back waist region 48, a front waist region 45, and crotch region 64 bordered by a pair of leg cuffs 50 and 52. The front waist region 45 includes the portion of the primary absorbent article 22 that when worn, is positioned on the front of the wearer while the back waist region 48 includes the portion of the primary absorbent article 22 that when worn is positioned on the back of the wearer. The crotch region 64 of the primary absorbent article 22 includes the portion of the primary absorbent article 22 that when worn is positioned between the legs of the wearer and covers the lower torso of the wearer. Each of the pair of leg cuffs 50 and 52 can be elasticized, if desired, by incorporating one or more strands of elastic, 54 and 56 respectively. The primary absorbent article 22 also has a liquid-permeable body-side liner 58, a liquid-impermeable outercover 60, and an absorbent core 62 positioned therebetween. Desirably, the absorbent core 62 is completely enclosed between the body-side liner 58 and the outercover 60. The body-side liner 58 is designed to allow rapid intake of body fluid. The body-side liner 58 can be made from the same types of materials as were described above for the body-side liner 12 in the secondary absorbent article 10. The body-side liner 58 can be formed from a woven material, nonwoven material, finely perforated film, net material, and so forth. Suitable materials include bonded carded webs of polyester, polypropylene, polyethylene, nylon or other heat-bondable fibers. Other polyolefins, such as copolymers of polypropylene and polyethylene, and linear low-density polyethylene also work well.

The liquid-impermeable outercover 60 functions to prevent body fluid from passing therethrough. The liquid-impermeable outercover 60 can be made from a microembossed polymeric film, such as polyethylene or polypropylene, or it can be made from bicomponent films. A desired material is a polyethylene film having a thickness of less than about 0.08 mm. Composite materials formed from a polymer film and a nonwoven fabric material can also be used. The composite sheets may be formed by extrusion of the polymer film onto a web of spunbond material to form an integral sheet. This material is desirable because the outer fabric surface is not irritating to the skin of the wearer and has a cushioned feel.

The primary absorbent article 22 has a crotch portion 64 formed between the pair of leg cuffs, 50 and 52 respectively. The primary absorbent article 22 is depicted as having a pair of refastenable ears 66 and 68 and a fastening zone 65. The article 22 can be positioned around the torso of the wearer and the refastenable ears 66 and 68 fastened to the fastening zone 65. Such a design is advantageous in allowing for tightening or loosening the primary absorbent article 22 around the waist of the wearer.

It should be noted that the primary absorbent article 22 could be in the form of a pant product, brief product, undergarment, or any other absorbent article. The primary absorbent article 22 is desirably a disposable or limited use product that will be discarded after a single use. Furthermore, the primary absorbent article 22 can be an absorbent article that has a pocket or fold for containing a disposable insert or liner. Any and all such absorbent articles 22 are capable of being used in conjunction with the secondary absorbent article 10.

The secondary absorbent article 10 can be visualized as a secondary absorbent structure that is used to extend or prolong the useful life of the primary absorbent article 22 and to enhance the skin-wellness of the wearer by providing a drier body-facing surface. Since the primary absorbent article 22 is more costly than the secondary absorbent article 10, the consumer can extend the life of the primary absorbent article 22 for a relatively small amount of money.

In use, the secondary absorbent article 10 is secured to at least a portion of the body-side liner 58 in the crotch portion 64 of the primary absorbent article 22 before the primary absorbent article 22 is placed on the body of the wearer. For the embodiment depicted in FIGS. 1-3, the releasable peel strip 46 is removed from the secondary absorbent article 10, exposing the attachment means 44. The crotch portion 20 of secondary absorbent article 10 is then placed or aligned over at least a portion of the crotch portion 64 of the primary absorbent article 22. Alternatively, the secondary absorbent article 10 may be placed in the area of the primary absorbent article 22 where it is most likely to be insulted by body fluid. The secondary absorbent article 10 is then pressed against the primary absorbent article 22 to secure the attachment means 44 thereto. Alternatively, the secondary absorbent article 10 may be secured to the primary absorbent article 22 by tucking one or more outer edges of the secondary absorbent article 10 inside a pocket or fold on the bodyfacing surface of the primary absorbent article 22. The primary absorbent article 22 can then be secured around the torso of the wearer.

In use, the secondary absorbent article 10 will acquire a cup shape configuration matching the crotch portion 64 of the primary absorbent article 22. A pair of upstanding side walls 70 and 72 forms a pair of reservoirs 74 and 76 adjacent to the absorbent core 16. The pair of reservoirs 74 and 76 are capable of retaining body fluid that has run off of the body-side liner 12 until the body fluid can be absorbed by the absorbent core 16. Thus, the pair of upstanding side walls 70 and 72 will assist in preventing side leakage of body fluid from the secondary absorbent article 10. Additionally, the optional containment flaps can be used to assist in preventing side leakage of body fluid from the secondary absorbent article 10.

It is possible to use two or more secondary absorbent articles 10 in the primary absorbent article 22 at the same time. Desirably, one secondary absorbent article 10 will be aligned vertically above the second secondary absorbent article 10. By using two or more secondary absorbent articles 10 at the same time, one can further extend or prolong the life of the primary absorbent article 22.

It should be noted that the purpose of the secondary absorbent article 10 is to receive and retain a portion of the bodily fluids, especially urine, discharged by the wearer. The secondary absorbent article 10 is constructed with a liquid-permeable body-side liner 12, an absorbent layer 30 having channels therethrough, and a liquid-permeable garment-side liner 14 so as to permit a substantial portion of bodily fluid discharged by the wearer to pass quickly down into the primary absorbent article 22. Because all of the discharged bodily fluid is not absorbed into the secondary absorbent article 10, the secondary absorbent article 10 stays dryer than if the secondary absorbent article 10 were to absorb all of the discharged bodily fluid. Since the secondary absorbent article 10 stays dryer for longer periods of time, skin wellness is promoted. Nonetheless, the overall liquid capacity of the combination of the primary absorbent article 22 and the secondary absorbent article 10 is greater to allow the combination to be worn for longer periods of time.

While the invention has been described in detail with respect to specific embodiments thereof, and particularly by the example described herein, it will be apparent to those skilled in the art that various alterations, modifications and other changes may be made without departing from the spirit and scope of the present invention. It is therefore intended that all such modifications, alterations and other changes be encompassed by the following claims.

We claim:

1. An absorbent article comprising a liquid-permeable liner attached to an absorbent core comprising an absorbent layer comprising a plurality of channels wherein the channels extend through the absorbent layer.

2. The absorbent article of claim 1 wherein the absorbent layer has a channel density between about 0.25 and about 20 channels per square centimeter.

3. The absorbent article of claim 1 wherein the absorbent layer has a channel density between about 1 channel per square centimeter and about 10 channels per square centimeter.

4. The absorbent article of claim 1 wherein the absorbent layer has a channel density between about 2 channels per square centimeter and about 5 channels per square centimeter.

5. The absorbent article of claim 1 wherein the channels have a cross-sectional area between about 1 and about 100 square millimeters.

6. The absorbent article of claim 1 wherein the total cross-sectional area of the channels at the external surface of the absorbent layer is between about 5% and about 30% of the external surface area of the absorbent layer.

7. The absorbent article of claim 1 wherein the absorbent core is enclosed by the liner.

8. The absorbent article of claim 1 wherein the liner comprises a spunbond material.

9. The absorbent article of claim 8 wherein the spunbond material has a basis weight between about 10 grams per square meter and about 34 grams per square meter.

10. The absorbent article of claim 1 wherein the absorbent core further comprises a tissue material wrapped about the absorbent layer.

11. The absorbent article of claim 1 wherein the channels are substantially parallel.

12. The absorbent article of claim 1 wherein the channels are substantially cylindrical.

13. The absorbent article of claim 12 wherein the channels have diameters between about 1 millimeter and about 10 millimeters.

14. The absorbent article of claim 12 wherein the channels have diameters between about 4 millimeters and about 6 millimeters.

15. The absorbent article of claim 1 wherein the absorbent layer further comprises hollow projections wherein the hollow projections form an extension of the channels.

16. An absorbent article comprising:
a first liquid-permeable liner;
a second liquid-permeable liner having an exterior surface with an attachment means secured thereto, the attachment means adapted to be adhered to a primary absorbent article, and;
an absorbent core comprising an absorbent layer comprising a plurality of channels wherein the channels extend through the absorbent layer, the absorbent layer has a channel density between about 1 and about 20 channels per square centimeter, the channels have a cross-sectional area between about 1 and about 25 square millimeters, and the total area of the channels does not exceed about 30% of the external surface area of the absorbent layer;
wherein the absorbent core is enclosed by the first and second liners.

17. The absorbent article of claim 16 wherein the attachment means comprises a hook fastener.

18. The absorbent article of claim 16 wherein the absorbent core further comprises a tissue material wrapped about the absorbent layer.

19. The absorbent article of claim 16 wherein the channels are substantially parallel.

20. An absorbent article comprising:
a first liquid-permeable liner;
a second liquid-permeable liner having an exterior surface with a garment adhesive secured thereto, the garment adhesive adapted to be adhered to a primary absorbent article, and;
an absorbent core comprising an absorbent layer comprising a plurality of channels wherein the channels extend through the absorbent layer, the absorbent layer has a channel density between about 1 and about 20 channels per square centimeter, the channels have cross-sectional areas between about 1 and about 25 square millimeters, and the total cross-sectional area of the channels is between about 5% and about 30% of the external surface area of the absorbent layer;
wherein the absorbent core is enclosed by the first and second liners to form an absorbent pad having a crotch portion.

21. The absorbent article of claim 20 wherein the absorbent article has a crotch width dimension that is less than about 100 millimeters.

22. The absorbent article of claim 20 wherein the absorbent core comprises at least two absorbent layers.

23. An absorbent article comprising:
a liquid-permeable body-side liner;
a liquid-permeable garment-side liner comprising a roughened surface, and;
an absorbent core comprising an absorbent layer comprising a plurality of channels wherein the channels extend through the absorbent layer;
wherein the absorbent core is enclosed by the body-side liner and the garment-side liner.

24. The absorbent article of claim 23 wherein the absorbent layer has a channel density between about 0.25 and about 20 channels per square centimeter.

25. The absorbent article of claim 23 wherein the channels have a cross-sectional area between about 1 and about 100 square millimeters.

26. The absorbent article of claim 23 wherein the total cross-sectional area of the channels at the external surface of the absorbent layer does not exceed about 30% of the external surface area of the absorbent layer.

27. The absorbent article of claim 23 wherein the cross-sectional areas of the channels at the external surface of the absorbent layer closest the body-side liner are greater than the cross-sectional areas of the channels at the external surface of the absorbent layer closest the garment-side liner.

28. The absorbent article of claim 23 wherein the channels are substantially parallel.

29. The absorbent article of claim 23 wherein the channels are substantially cylindrical.

30. The absorbent article of claim 29 wherein the channels have diameters between about 1 millimeter and about 10 millimeters.

31. The absorbent article of claim 23 wherein the absorbent layer further comprises hollow projections wherein the hollow projections form an extension of the channels.

32. An absorbent article comprising:
a liquid-permeable body-side liner;
a liquid-permeable garment-side liner, and;
an absorbent core comprising:
a first absorbent layer comprising a plurality of channels wherein
the channels extend through the first absorbent layer, and;
a second absorbent layer;
wherein the absorbent core is enclosed by the body-side liner and the garment-side liner.

33. The absorbent article of claim 32 wherein the first absorbent layer has a channel density between about 0.25 and about 20 channels per square centimeter.

34. The absorbent article of claim 32 wherein the channels have a cross-sectional area between about 1 and about 100 square millimeters.

35. The absorbent article of claim 32 wherein the total cross-sectional area of the channels at the external surface of the first absorbent layer does not exceed about 30% of the external surface area of the first absorbent layer.

36. The absorbent article of claim 32 wherein the channels are substantially parallel.

37. The absorbent article of claim 32 wherein the channels are substantially cylindrical.

38. The absorbent article of claim 37 wherein the channels have diameters between about 1 millimeter and about 10 millimeters.

39. The absorbent article of claim 32 wherein the first absorbent layer further comprises hollow projections wherein the hollow projections form an extension of the channels.

40. An absorbent article comprising:
a liquid-permeable body-side liner;
a liquid-permeable garment-side liner, and;
an absorbent core comprising:
a first absorbent layer comprising wood fluff defining a plurality of channels wherein the channels extend through the first absorbent layer, and;
a second absorbent layer comprising wood fluff and superabsorbent material;
wherein the absorbent core is enclosed by the body-side liner and the garment-side liner.

41. The absorbent article of claim 40 wherein the first absorbent layer has a channel density between about 0.25 and about 20 channels per square centimeter.

42. The absorbent article of claim 40 wherein the channels have a cross-sectional area between about 1 and about 100 square millimeters.

43. The absorbent article of claim 40 wherein the total cross-sectional area of the channels at the external surface of the first absorbent layer does not exceed about 30% of the external surface area of the first absorbent layer.

44. The absorbent article of claim 40 wherein the channels are substantially parallel.

45. The absorbent article of claim 40 wherein the channels are substantially cylindrical.

46. The absorbent article of claim 45 wherein the channels have diameters between about 1 millimeter and about 10 millimeters.

47. The absorbent article of claim 40 wherein the first absorbent layer further comprises hollow projections wherein the hollow projections form an extension of the channels.

48. An absorbent system comprising:
a primary absorbent article; and,
a secondary absorbent article attached to the primary absorbent article, the secondary absorbent article comprising a liquid-permeable liner attached to an absorbent core comprising an absorbent layer comprising a plurality of channels wherein the channels extend through the absorbent layer.

49. The absorbent system of claim 48 wherein the absorbent layer has a channel density between about 0.25 and about 20 channels per square centimeter.

50. The absorbent system of claim 48 wherein the absorbent layer has a channel density between about 1 channel per square centimeter and about 10 channels per square centimeter.

51. The absorbent system of claim 48 wherein the absorbent layer has a channel density between about 2 channels per square centimeter and about 5 channels per square centimeter.

52. The absorbent system of claim 48 wherein the channels have a cross-sectional area between about 1 and about 100 square millimeters.

53. The absorbent system of claim 48 wherein the total cross-sectional area of the channels at the external surface of the absorbent layer is between about 5% and about 30% of the external surface area of the absorbent layer.

54. The absorbent system of claim 48 wherein the absorbent core is enclosed by the liner.

55. The absorbent system of claim 48 wherein the liner comprises a spunbond material.

56. The absorbent system of claim 48 wherein the absorbent core further comprises a tissue material wrapped about the absorbent layer.

57. The absorbent system of claim 48 wherein the channels are substantially parallel.

58. The absorbent system of claim 48 wherein the channels are substantially cylindrical.

59. The absorbent system of claim 58 wherein the channels have diameters between about 1 millimeter and about 10 millimeters.

60. The absorbent system of claim 58 wherein the channels have diameters between about 4 millimeters and about 6 millimeters.

61. The absorbent system of claim 48 wherein the absorbent layer further comprises hollow projections wherein the hollow projections form an extension of the channels.

62. The absorbent system of claim 48 wherein the secondary absorbent article is substantially the same shape as the primary absorbent article.

63. The absorbent system of claim 48 wherein the absorbent capacity of the primary absorbent article substantially matches the absorbent capacity of the secondary absorbent article.
